(19) United States
(12) Patent Application Publication
(10) Pub. No.: US 2021/0358056 A1
(43) Pub. Date: Nov. 18, 2021

(54) MULTI-PLATFORM SOCIAL MEDIA NETWORK

(71) Applicant: Azeem Khan, Forest Hills, NY (US)

(72) Inventor: Azeem Khan, Forest Hills, NY (US)

(21) Appl. No.: 17/318,853

(22) Filed: May 12, 2021

Related U.S. Application Data
(60) Provisional application No. 63/023,309, filed on May 12, 2020.

(51) Int. Cl. G06Q 50/00; H04L 29/08; G06Q 30/02; G06Q 30/00

(52) U.S. Cl. CPC G06Q 50/01; H04L 67/306; G06Q 50/2057; G06Q 30/018; G06Q 30/0282

(57) ABSTRACT

A multi-platform social media platform, which includes an adaptive system for ranking user credibility based on feedback from other users. A user ranking corresponds to a user's initial ranking and the feedback that is provided by other users over time. The user's initial ranking is assigned by the social media platform based on the user's professional qualifications and education. The feedback provided from other users changes the user's ranking, based on whether the feedback is positive or negative. The user's rank is used as an analog for the user's credibility and the trustworthiness of content created by the user. Users of the social media platform may use a user's rank to determine whether the user is a credible source of information.

### RELATED U.S. APPLICATION DATA

This claims the benefit of U.S. Provisional Patent Application No. 63/023,309 filed May 12, 2020, which is hereby incorporated by reference herein.

### FIELD OF THE INVENTION

The present invention relates to a method and system of connecting users of a social media network that have a shared interest in certain medical conditions, and ranking the credibility of social media users and their content. The invention may be used in such social media platforms involving people with similar interests, people with similar medical conditions, medical providers having similar specialties, for connecting people with medical conditions to medical providers for those conditions and connecting people with similar interests.

### BACKGROUND OF THE INVENTION

Social media and mobile applications have increased connectivity between wider peer groups, enabling any user to communicate with a network of other users, from anywhere in the world. There are many different types of social media networks for many different types of interests. There are many methods of connecting social media users with various interests, and many different methods of communication between social media users.

For example, U.S. Pat. No. 7,188,153 issued Mar. 6, 2007 to Lunt, et al. discloses a system and method for managing connections in an online social network. The online social network collects descriptive data about various individuals and allows those individuals to indicate other individuals with whom they have a personal relationship. The descriptive data and the relationship data are integrated and processed to reveal the series of social relationships connecting any two individuals within a social network. A maximum degree of separation setting is provided and set to at least two. The maximum degree of separation setting limits the amount of searching that is carried out when searching for a connection between two individuals in the social network. Users in the system have the ability to provide information about one another. For example, one user might write a positive comment about a second user's personality. In one implementation, the second user may accept or reject display of the comment. Further, in this implementation, any other users who are connected to the second user through any number of acquaintance pathways may see the comment as part of the “descriptive data” about the second user.

U.S. Pat. No. 7,117,254 issued Oct. 3, 2006 to Lunt, et al. discloses a method of inducing content uploads in an online network includes the steps of storing content relating to a first member of the network that is submitted by a second member of the network, receiving approval of the content from the first member, and associating the content with the first member. The uploaded content may comprise an image file containing a photo of the first member and a caption associated with the photo image. The second user uploads the content relating to the first member using a graphic user interface (GUI). This GUI is made available to the second user through a hyperlink that appears when a profile page of the first member is accessed if the degree of separation between the first member and the second member is less than or equal to a maximum degree of separation set by the first user. On the other hand, the hyperlink to this GUI will not appear when the profile page of the first member is accessed by the second member if the degree of separation between the first member and the second member is greater than the maximum degree of separation set by the first user. The default setting for this maximum degree of separation is one. US Patent Application publication No. 20090320101 published Dec. 24, 2009 (inventors: Doyle, III, et al.) discloses a system and method that authenticate the identity of the person behind a username and store that information in a manner that allows a first person communicating on a social network with a second person to confirm that the identity of the second person is known and authenticate without requiring the second person to reveal identity information (other than their user name/screen name) to the first person and vice versa.

US Patent Application Publication No. 20110299478 published Dec. 8, 2011 (Inventor: Clark) discloses a method for initiating communication between at least two users of two or more users operating communications devices, at least one of which is a cellular telephone device, on a local area network (LAN) connected to a cellular telephone network having gateway access to the Internet network, the communication advanced according to priority profile exchange and matching between the users. The method comprises (a) activating the communications devices to exchange profile information with each other; (b) receiving profiles, each communications device receiving profiles from other participating communications devices, including the cellular telephone receiving profiles from a Web server in the Internet; (c) comparing the received profiles to local profiles stored on each of the participating communications devices or on the Web server in the Internet network, the matching performed according to priority criteria; (d) registering at least one match of profiles on at least one of the communications devices or on the Web server; (e) identifying the sending device of the highest priority profile matched either at the local device or at the Web server; and (f) initiating text messaging communication between the communications devices involved in the profile match, the communication initiated from the device registering the profile match or by the Web server.

U.S. Pat. No. 8,209,310 issued Jun. 26, 2012 to Metcalfe discloses a professional networking system and method that facilitate a meeting between two or more users of the system for the purpose of expanding the users' professional networks. Users are matched by the system based on one or more available meeting dates of each user, and one or more sought characteristics corresponding to one or more characteristics of the second user that the first user profile seeks in another user. The system does not directly reveal the characteristics of one matched user to any other user except certain basic contact information may be disclosed to another matched user to facilitate a meeting.

Many other systems and methods similar to those described above exist in the art. In general, however, they do not provide a sufficient degree of accuracy identifying people of similar interests nor do they facilitate communications between trained professionals and individuals who might benefit from the opinions and such trained professionals.

The purpose behind the application is multifaceted. Firstly, it is the present invention's intention to foster a safe and casual environment for the user to express interest in a certain topic or medical condition. A user may post their thoughts, opinions, images or videos related to certain topics, such as a chronic medical condition. Secondly, it is the present invention's intention to foster connections between social media network users with similar interests. Thirdly, it is the present invention's intention to provide a system and method for ranking social media users', and their content's, credibility in a systemic manner, wherein a user's rank changes over time based on the feedback received from other users.

### SUMMARY OF THE INVENTION

The purpose behind the application is multifaceted. Firstly, it is the present invention's intention to foster a safe and educational environment for a user to learn about various medical topics. The user can use a variety of cloud-based platforms, including apps for mobile devices, to access curated information and non-curated information about various medical topics. The curated information is included to help users reduce the risk of accessing erroneous medical information. An element of the curation process includes among other things a system and method for peer feedback and peer ranking for identifying high quality information and users, based on a combination of user qualifications and the peer feedback that is given by other users. Secondly, it is the present invention's intention to facilitate communication between users by assisting users to find and communicate with other users that have similar interests. A user may search through user profiles and key words to find other users with similar interests.

### BRIEF DESCRIPTION OF THE DRAWINGS

FIG. 1 is a flow-chart that illustrates the sequence of events taking place in the method of the invention for the communication between users with common interests.

FIG. 2 is a flow-chart that illustrates the sequence of events taking place in the method of the invention for the Professional Users receiving positive and negative feedback.

FIG. 3 is a flow-chart that illustrates the sequence of events taking place in the method of the invention for the General Users receiving positive and negative feedback.

### DETAILED DESCRIPTION OF THE INVENTION

It is an object of the present invention to create a multi-user, multi-platform, social network and mobile application which connects users with similar interests across multiple platforms. Another object of the present invention is to provide a system and method for peer rating, vetting, validating, review, and credentialing of users and user content.

It is an object of the present invention to create a multi-user, multi-platform, social networking and mobile application which connects users with shared interests, and interest groups. The present invention can connect various users to other users with similar interests and facilitate communication between two or more separate users via various methods of real time and non-real time communication systems, and an application environment encouraging the social connectivity and communication between two or more separate users with similar interests.

It is an object of the present invention to create a system and method of peer rating, vetting, validating, reviewing and credentialing users and user content on a multi-user, multi-platform, social network and mobile application (collectively the “Site”). The present invention allows users to review and rate other users, and their content. The present invention includes a system consisting of multiple classes of users including without limitation, “General Users” and “Professional Users.” General Users are defined as an average person who joins the Site. “Professional Users” are defined as users that join the Site, who are professionals or seen as highly knowledgeable or experienced in a particular field. For example, in the healthcare field, Professional Users would include medical doctors, medical researchers, scientists and nurses. General Users (“GU” or “GUs” for the plural of GU) and Professional Users (“PU” or “PUs” for the plural of PU) (each a “User”) are different User classes or classifications. When a User signs up to the site that User is given an initial Peer Rank number based on that User's class or classification (“Class”). For example, a GU might receive a Peer Rank of “100” while a PU would initially receive a PU of “1000.” A “Peer Rank” (“PR” or “PRs” for the plural of PR) is defined as a number assigned to a User when they join the Site, which is designed to reflect the User's background knowledge, credentials and qualifications in a field. Over time a PR will increase or decrease based on feedback, of User created content, given by other Users. If a User receives positive feedback, from another User, then their PR will increase. If a User receives negative feedback, from another User, then a User's PR will decrease. Initially, PUs are assigned higher PRs than GUs, to reflect their increased knowledge, education and experience in an applicable field. As GU's and PU's create and post content or messages on the Site the GU and PU may receive positive or negative feedback from either GUs or PUs, this feedback is defined as “Peer Feedback,” which may be either positive or negative responses to content or messages posted by a User. Peer Feedback is provided to Users through a feedback mechanism on the Site, which allows a user to provide Peer Feedback. Whenever any content or messages are posted through the Site by a User, such content or messages will include a mechanism, for example a clickable button, that allows Users to provide Peer Feedback. The Weight given to Peer Feedback by one User classification may not be equal to the Weight given to Peer Feedback by a different User classification or by a different User. “Weight” is defined as the amount that Peer Feedback changes a User's PR in either a positive or negative direction. The Weight given to Peer Feedback is determined by the PR and classification of the User providing the Peer Feedback. The greater a User's PR the greater the Weight of that User's Peer Feedback will be on the recipient User of Peer Feedback. For example, a User that has a PR of “5000” will be able to affect another User's PR with a single Peer Feedback response in a greater manner than a Peer Feedback from a User having a PR of “100.” The Peer Feedback system is structured so that it gives Users, which have the greatest experience, education, training and credibility, more influence in determining which other Users are creating, or posting, credible content by making Peer Feedback, which is created by User's with high PR, more Weight in adjusting the PR of other users. PUs, or highly ranked GUs, are given more authority, within the Peer Feedback system, to determine whether User created or posted content is legitimate, trustworthy and credible. The Peer Feedback system and method acts as mechanism to allow Site Users to internally peer review and establishing the credibility of User created Site content and of Users. The Peer Feedback system assists all Users in understanding which Users are trustworthy and credible content creators, or respected by other Users. The Peer Feedback system also includes a method for Site administration oversight of PRs. The Site administration shall have the authority to change any Users PR at any time. This oversight is an invaluable part of the Peer Feedback system, in that it allows the Site administration to correct any erroneous, manipulated or arbitrary PRs.

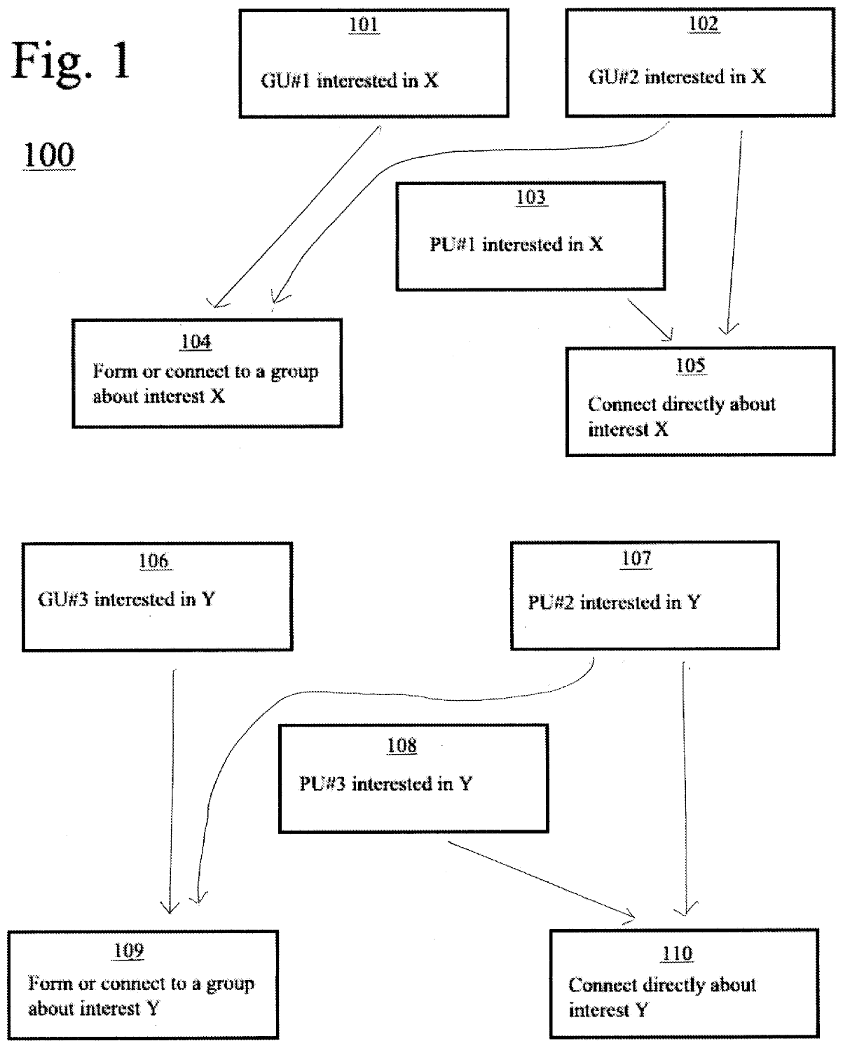

FIG. 1. is a flow chart 100 illustrating a process of connecting users of the multi-platform social media network, who are interested in the same topics. Flow Chart 100 illustrates how that GU#1 is interested in topic X 101, GU#2 is also interested in topic X 102, and PU#1 is interested in topic X 103. GU#1 and GU#2 form or connect to a group of people interested in topic X 104. Additionally, GU#2 may connect with PU#1 directly to communicate regarding their interest in topic X 105. Flow chart 100, further illustrates that GU#3 is interested in topic Y 106, PU#2 is interested in topic Y 107, and PU#3 is interested in topic Y 108. GU#3 and PU#2 may form or connect to a group to communicate regarding their interest in topic Y 109. Additionally, PU#2 and PU#3 may connect directly to communicate regarding their interest in topic Y 110.

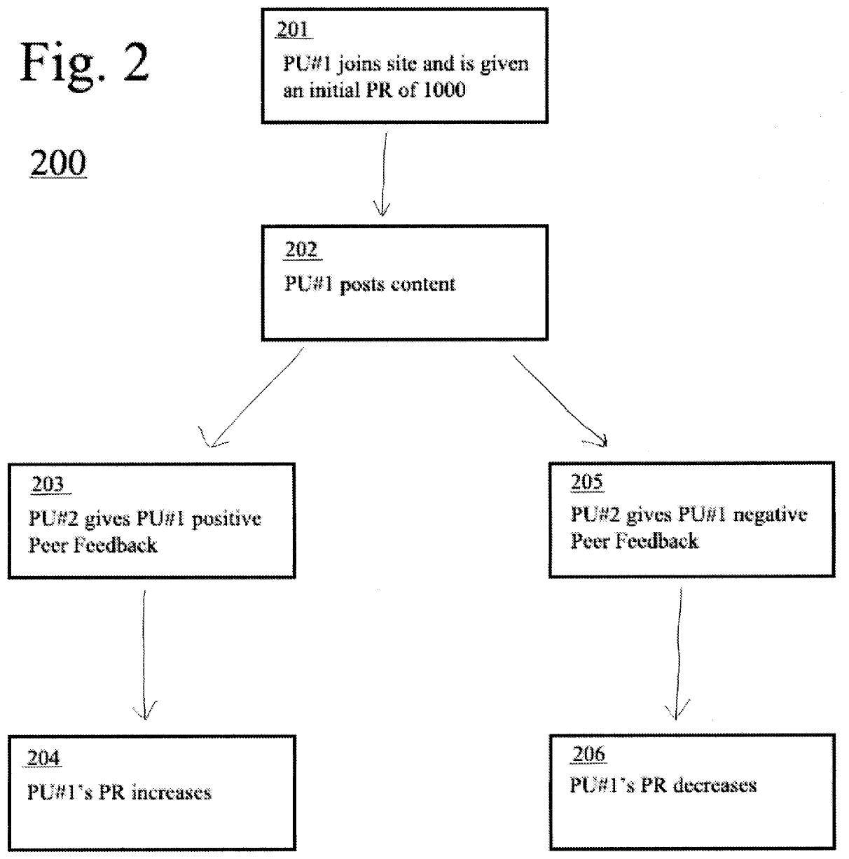

FIG. 2. is a flow chart 200 illustrating a process of the Peer Feedback system working to alter the PR of PU#1. PU#1 joins the Site and is given an initial PR of 1000 201. PU#1 then creates and posts content to the Site about a topic 202. PU#1 then receives positive Peer Feedback from PU#2 203. PU#1's PR increases as a result of PU#2's positive Peer Feedback 204. Alternatively, after PU#1 creates and posts content to the Site about a topic 202, PU#1 may receive negative Peer Feedback from PU#2 205. As a result of the negative Peer Feedback from PU#2, PU#1's PR decreases.

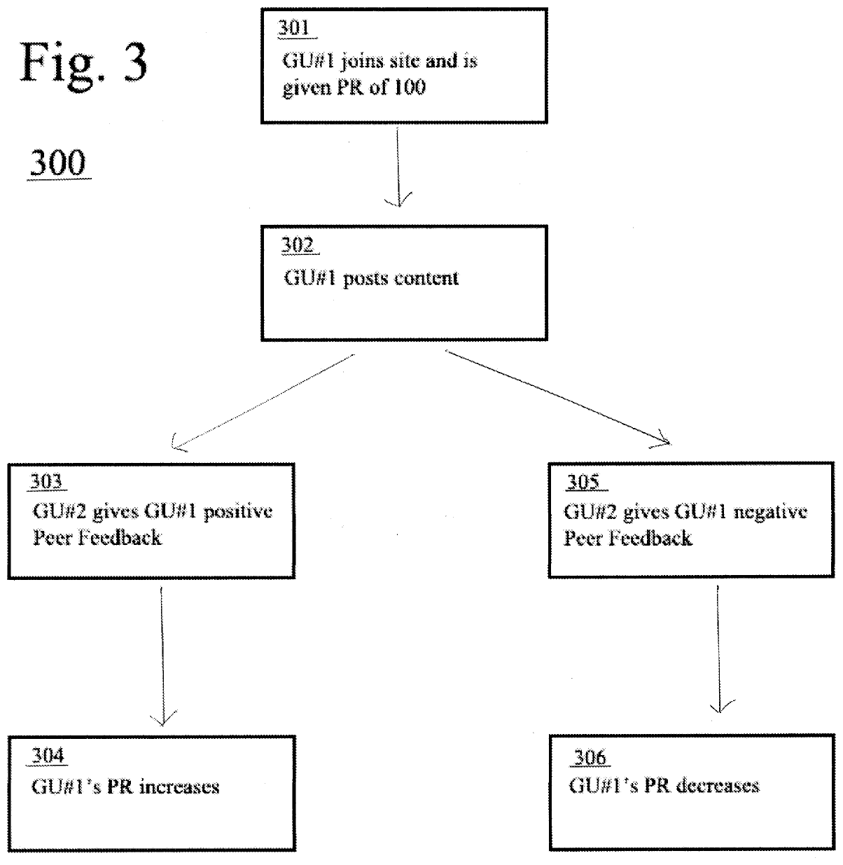

FIG. 3. is a flow chart 300 illustrating a process of the Peer Feedback system working to alter the PR of GU#1. GU#1 joins the Site and is given an initial PR of 100 301. GU#1 then creates and posts content to the Site 302. GU#2 then gives GU#1 positive Peer Feedback 303. GU#1's PR increases as a result of the positive Peer Feedback from GU#2. Alternatively, if GU#2 gives GU#1 negative Peer Feedback 305, then GU#1's PR will decrease 306.

The present invention can be applied across a wide variety of social networks and is not limited mobile applications for healthcare topics.

What sets this application apart from other social network sites is the use of curated and non-curated information through a peer feedback and peer rank system and method, as well as, the manner in which users search for other users based on their aligned interests. PRs are dynamic and change over time based on peer feedback from other users.

The present invention's mobile application features a method and system for rating users to establish the average trustworthiness of a user and a user's content in the social network. Mobile users are provided with a feature that allows them to rate a user's private messages as well as content posted by users, including without limitation, image posts, video posts, and written posts.

Thus, according to one aspect, the method of the invention for rating the trustworthiness of social media network users can be summarized as follows: (a) assigning different classes of users with initial ranks; (b) increasing, or decreasing, a user's rank based on positive, or negative, feedback from another users (the “Feedback User”), the size of the increase, or decrease, in rank is based on the class and rank of the Feedback User at the time when feedback is given; (c) feedback is given by a Feedback User if the Feedback User likes or dislikes a post, message or content posted by a user, or because the Feedback User finds a post, message or content to be trustworthy or untrustworthy. A user's rank provides other users with guidance regarding that user's trustworthiness and whether Feedback Users liked or disliked that user's messages, content and posts.

What is claimed is:

1. A method of peer rating users on a social media network, the method implemented by a processor of a computing system, comprising:
assigning, in real time by the processor, a user's rank based on the user's background; and
adjusting, in real time by the processor, the user's rank based on a feedback from another user.

2. The method as recited in claim 1 wherein the user is assigned a class, in real time by the processor.

3. The method as recited in claim 1 wherein the user's rank is increased, in real time by the processor, as a result of receiving a positive feedback from another user.

4. The method as recited in claim 1 wherein the user's rank is decreased in real time by the processor, as a result of receiving a negative feedback from another user.

5. The method as recited in claim 1 wherein the user's rank is initially assigned based on the user's education.

6. The method as recited in claim 1 wherein the user's rank is initially assigned based on the user's career training.

7. The method as recited in claim 1 wherein the user's rank is initially assigned based on the user having a state or federal license.

8. The method as recited in claim 2 wherein the user's class is initially assigned based on the user's career training.

9. The method as recited in claim 2 wherein the user's class is initially assigned based on the user having a state or federal license.
